(19) United States
(12) Patent Application Publication
(10) Pub. No.: US 2026/0174658 A1
(43) Pub. Date: Jun. 25, 2026

(54) SUNSCREEN COMPOSITION

(71) Applicant: Kenvue Brands LLC, Summit, NJ (US)

(72) Inventors: Sonia Milojevic Teran, Princeton, NJ (US); Marina Teles Sutija, North Plainfield, NJ (US); Tatiana Luts, Bridgewater, NJ (US); Connor F. DeVoe, Morristown, NJ (US)

(21) Appl. No.: 19/419,433

(22) Filed: Dec. 15, 2025

Related U.S. Application Data
(60) Provisional application No. 63/736,279, filed on Dec. 19, 2024.

(51) Int. Cl. A61K 8/55; A61K 8/06; A61K 8/25; A61K 8/34; A61K 8/67; A61K 8/73; A61K 8/81; A61K 8/92; A61Q 17/04

(52) U.S. Cl. CPC A61K 8/55; A61K 8/062; A61K 8/25; A61K 8/345; A61K 8/675; A61K 8/676; A61K 8/678; A61K 8/732; A61K 8/735; A61K 8/8152; A61K 8/8182; A61K 8/922; A61Q 17/04; A61K 2800/26; A61K 2800/54

(57) ABSTRACT

The present invention provides a sunscreen composition comprising: (i) an organic UV filter selected from the group consisting of avobenzone, homosalate, octocrylene, octisalate, bis-ethylhexyloxyphenol methoxyphenyl triazine, and combinations thereof; (ii) a polymeric emulsifier selected from the group consisting of sodium acryloyldimethyltaurate/VP crosspolymer, acrylates/C10-30 alkyl acrylate crosspolymer, and combinations thereof; (iii) a solid absorbent selected from the group consisting of silica, aluminum starch octenylsuccinate, and combinations thereof; and (iv) trilaureth-4 phosphate.

This application claims the benefit of U.S. provisional application 63/736,279 filed on Dec. 19, 2024, the complete disclosure of which is hereby incorporated herein by reference for all purposes.

The present invention provides a sunscreen composition comprising: (i) a UV absorber selected from the group consisting of avobenzone, homosalate, octocrylene, octisalate, bis-ethylhexyloxyphenol methoxyphenyl triazine, and combinations thereof; (ii) a polymeric emulsifier selected from the group consisting of sodium acryloyldimethyltaurate/vinylpyrrolidine crosspolymer, acrylates/C10-30 alkyl acrylate crosspolymer, and combinations thereof; (iii) a solid absorbent selected from the group consisting of silica, aluminum starch octenylsuccinate, and combinations thereof; and (vi) trilaureth-4 phosphate.

### BACKGROUND OF THE INVENTION

It is well known that prolonged exposure to ultraviolet radiation, such as from the sun, can lead to the formation of light dermatoses and erythemas, and increase the risk of skin cancers, such as melanoma. Exposure to UV radiation also accelerates skin aging, such as loss of skin elasticity and wrinkling.

For these reasons, sunscreen compositions are commonly used to provide protection from the sun, and a variety of sunscreen compositions are commercially available. Sunscreen compositions may contain a combination of organic UV absorbers such as homosalate, octocrylene, and octisalate, which are liquids at room temperature, along with solid UV absorbers such as avobenzone and bis-ethylhexyloxyphenol methoxyphenyl triazine, and may be in the form of oil-in-water emulsions to provide good aesthetic properties. In addition to UV absorbers it is desirable to include oil-absorbing solids in such formulations to create a desirable mattifying effect or to provide a pleasant skin feel. However, it is often difficult to stabilize compositions containing liquid UV absorbers, solid UV absorbers, and other solids, for example silica or aluminum starch octenylsuccinate.

US-2019-0015317AI relates to sunscreen compositions comprising at least about 10 weight percent of one or more UV filters, a hydrophobically modified polyurethane, a viscosity increasing polymer, and glyceryl stearate. The viscosity increasing polymer may be a taurate copolymer, such as sodium acryloyldimethyltaurate/vinylpyrrolidine crosspolymer, or an acrylate crosspolymer, such as acrylates/C10-30 alkyl acrylate crosspolymer. The composition may also comprise silica or glycerin. This composition has surprisingly light aesthetics while providing excellent UV protection.

However, alternative sunscreen formulations are still required.

Applicants have now discovered sunscreen compositions that are oil-in-water emulsions comprising avobenzone, homosalate, octocrylene, octisalate, and/or bis-ethylhexyloxyphenol methoxyphenyl triazine, solid absorbents, and a combination of a polymeric emulsifier with trilaureth-4 phosphate. Surprisingly, the combination of polymeric emulsifier with trilaureth-4 phosphate allows for the formation of stable emulsions containing liquid organic UV filters, solid organic UV filters, and solid absorbents. These compositions are fluid, oil-in-water emulsions that advantageously provide in vivo or in vitro SPF levels of at least about 30, preferably at least about 40, more preferably at least about 50. As fluid emulsions, they also have good aesthetics as lotions. In certain embodiments, these sunscreen compositions are very lightweight and fast absorbing, a difficult aesthetic to achieve in a sunscreen composition with high loads of UV filters.

### SUMMARY OF THE INVENTION

The present invention provides a sunscreen composition comprising: (i) an organic UV filter selected from the group consisting of avobenzone, homosalate, octocrylene, octisalate, bis-ethylhexyloxyphenol methoxyphenyl triazine, and combinations thereof; (ii) a polymeric emulsifier selected from the group consisting of sodium acryloyldimethyltaurate/vinylpyrrolidine crosspolymer, acrylates/C10-30 alkyl acrylate crosspolymer, and combinations thereof; (iii) a solid absorbent selected from the group consisting of silica, aluminum starch octenylsuccinate, and combinations thereof; and (vi) trilaureth-4 phosphate; wherein the composition is an oil-in-water emulsion.

The above composition may comprise: (i) up to 5 weight % of avobenzone based on the total weight of the composition; (ii) up to 15 weight % of homosalate based on the total weight of the composition; (iii) up to 10 weight % of octocrylene based on the total weight of the composition; (iv) up to 5 weight % of octisalate based on the total weight of the composition; (v) up to 6 weight % of bis-ethylhexyloxyphenol methoxyphenyl triazine based on the total weight of the composition; (vi) about 0.01 to about 1.0 weight % of polymeric emulsifier based on the total weight of the composition; (vii) about 2 to about 15 weight % of solid absorbent based on the total weight of the composition; and (viii) about 0.75 to about 3 weight % of trilaureth-4 phosphate based on the total weight of the composition.

The present invention also provides a sunscreen composition comprising: (i) about 3 weight % of avobenzone based on the total weight of the composition; (ii) about 15 weight % of homosalate based on the total weight of the composition; (iii) about 10 weight % of octocrylene based on the total weight of the composition; (iv) about 5 weight % of octisalate based on the total weight of the composition; (v) about 0.2 weight % of acrylates/C10-30 alkyl acrylate crosspolymer based on the total weight of the composition; (vi) about 7 weight % of silica based on the total weight of the composition; (vii) about 5 weight % of aluminum starch octenylsuccinate based on the total weight of the composition; and (viii) about 2.5 weight % of trilaureth-4 phosphate based on the total weight of the composition; wherein the composition is an oil-in-water emulsion.

The present invention also provides a sunscreen composition comprising: (i) about 3 weight % of avobenzone based on the total weight of the composition; (ii) about 13.5 weight % of homosalate based on the total weight of the composition; (iii) about 10 weight % of octocrylene based on the total weight of the composition; (iv) about 5 weight % of octisalate based on the total weight of the composition; (v) about 0.5 weight % of sodium acryloyldimethyltaurate/vinylpyrrolidine crosspolymer based on the total weight of the composition; (vi) about 2.5 weight % of silica based on the total weight of the composition; (vii) about 1 weight % of aluminum starch octenylsuccinate based on the total weight of the composition; (viii) about 2 weight % of trilaureth-4 phosphate based on the total weight of the composition; and (ix) about 3.5 weight % of glycerin based on the total weight of the composition; wherein the composition is an oil-in-water emulsion.

The present invention further provides a sunscreen composition comprising: (i) about 3 weight % of avobenzone based on the total weight of the composition; (ii) about 5 weight % of octisalate based on the total weight of the composition; (iii) about 6 weight % of bis-ethylhexyloxyphenol methoxyphenyl triazine based on the total weight of the composition; (iv) about 0.45 weight % of sodium acryloyldimethyltaurate/vinylpyrrolidine crosspolymer based on the total weight of the composition; (v) about 2 weight % of silica based on the total weight of the composition; (vi) about 1 weight % of aluminum starch octenylsuccinate based on the total weight of the composition; and (vii) about 2 weight % of trilaureth-4 phosphate based on the total weight of the composition; wherein the composition is an oil-in-water emulsion.

The present invention further provides a sunscreen composition comprising: (i) about 3 weight % of avobenzone based on the total weight of the composition; (ii) about 5 weight % of octisalate based on the total weight of the composition; (iii) about 15 weight % of homosalate based on the total weight of the composition; (iv) about 6 weight % of bis-ethylhexyloxyphenol methoxyphenyl triazine based on the total weight of the composition; (v) about 0.5 weight % of sodium acryloyldimethyltaurate/vinylpyrrolidine crosspolymer based on the total weight of the composition; (vi) about 2 weight % of silica based on the total weight of the composition; (vii) about 1 weight % of aluminum starch octenylsuccinate based on the total weight of the composition; and (viii) about 2 weight % of trilaureth-4 phosphate based on the total weight of the composition; wherein the composition is an oil-in-water emulsion.

### DETAILED DESCRIPTION OF THE INVENTION

Unless defined otherwise, all technical and scientific terms used herein have the same meaning as commonly understood by one of ordinary skill in the art to which the invention belongs. All publications, patent applications, patents, and other references mentioned herein are incorporated by reference.

As used herein, “topically applying” means directly laying on or spreading on outer skin or the scalp, e.g., by use of the hands or an applicator such as a wipe, roller, or spray.

As used herein, “cosmetic” refers to a beautifying substance or preparation which preserves, restores, bestows, simulates, or enhances the appearance of bodily beauty or appears to enhance the beauty or youthfulness, specifically as it relates to the appearance of tissue or skin.

As used herein, “sunscreen composition” means a formulation that is a lotion that absorbs and/or reflects some of the sun's ultraviolet (UV) radiation and thus helps protect against negative effects of sun exposure, e.g., sunburn, premature aging, etc.

As used herein, “cosmetically effective amount” means an amount of a physiologically active compound or composition sufficient for treating one or more conditions, but low enough to avoid serious side effects. The cosmetically effective amount of the compound or composition will vary with the condition being treated, the age and physical condition of the end user, the severity of the condition being treated/prevented, the duration of the treatment, the nature of other treatments, the specific compound or product/composition employed, the cosmetically acceptable carrier utilized, and like factors.

As used herein, “cosmetically acceptable” means that the ingredients the term describes are suitable for use in contact with tissues (e.g., the skin) without undue toxicity, incompatibility, instability, irritation, allergic response, or the like.

As used herein, a “cosmetically acceptable active agent” is a compound (synthetic or natural) that has a cosmetic or therapeutic effect on the skin.

As used herein, “treatment or treating” refers to mitigating, reducing, preventing, improving, or eliminating the presence or signs of a condition or disorder.

As used herein, “substantially free of” means the ingredient referred to is not directly or intentionally added. Preferably, “substantially free of” means containing less than about 1% by weight. More preferably “substantially free of” means containing less than about 0.5% by weight. Even more preferably “substantially free of” means containing less than about 0.1% by weight. The present sunscreen composition may be completely free of an ingredient, i.e., contain none of the ingredient.

As used herein, “UV absorber” means an active ingredient whose specific function is to absorb radiation in some portion of the ultraviolet spectrum (290 nm-400 nm), such as one having an extinction coefficient of at least about 1000 mol⁻¹ cm⁻¹, for at least one wavelength within the above-defined ultraviolet spectrum. UV absorbers include for example approved sun filters and are distinguished from compounds having other functions that also happen to absorb UV radiation.

As used herein, “UV blocker” means an active ingredient whose specific function is to reflect or scatter UV radiation, for example mineral sunscreens such as titanium dioxide or zinc oxide.

As used herein, “phase-stable” means the water and oil phases of an emulsion do not appreciably separate at room temperature for at least two weeks.

Unless otherwise indicated, a percentage or concentration refers to a percentage or concentration by weight (i.e., % (W/W) based on the total weight of the composition. Unless stated otherwise, all ranges are inclusive of the endpoints, e.g., “from 4 to 9” includes the endpoints 4 and 9.

### UV Absorbers

The sunscreen composition of the invention comprises an organic UV absorber selected from the group consisting of avobenzone, homosalate, octocrylene, octisalate, and bis-ethylhexyloxyphenol methoxyphenyl triazine. The composition may comprise a combination of two or more of avobenzone, homosalate, octocrylene, octisalate, and bis-ethylhexyloxyphenol methoxyphenyl triazine.

Preferably, the composition is substantially free, or completely free, of oxybenzone.

The composition may comprise 0 to about 10% by weight of avobenzone based on the total weight of the composition. Preferably, the composition comprises about 3% by weight of avobenzone based on the total weight of the composition.

The composition may comprise 0 to about 15% by weight of homosalate based on the total weight of the composition. Preferably, the composition comprises about 13 to about 15% by weight of homosalate based on the total weight of the composition.

The composition may comprise 0 to about 12% by weight of octocrylene based on the total weight of the composition. Preferably, the composition comprises about 10% by weight of octocrylene based on the total weight of the composition.

The composition may comprise 0 to about 10% by weight of octisalate based on the total weight of the composition. Preferably, the composition comprises about 5% by weight of octisalate based on the total weight of the composition.

The composition may comprise 0 to about 6% by weight of bis-ethylhexyloxyphenol methoxyphenyl triazine. Preferably, the composition comprises about 6% by weight of bis-ethylhexyloxyphenol methoxyphenyl triazine.

In one embodiment, the composition comprises about 3% by weight avobenzone, about 15% by weight homosalate, about 10% by weight octocrylene, and about 5% by weight octisalate.

In another embodiment, the composition comprises about 3% by weight avobenzone, about 13.5% by weight homosalate, about 10% by weight octocrylene, and about 5% by weight octisalate.

In another embodiment, the composition comprises about 3% by weight avobenzone, about 5% by weight octisalate, and about 6% by weight bis-ethylhexyloxyphenol methoxyphenyl triazine.

In another embodiment, the composition comprises about 3% by weight avobenzone, about 5% by weight octisalate, about 15% homosalate, and about 6% by weight bis-ethylhexyloxyphenol methoxyphenyl triazine.

Avobenzone is commercially available, for example under the trade name Neo Heliopan 357 from Symrise.

Homosalate is commercially available, for example under the trade name Neo Heliopan HMS from Symrise.

Octocrylene is commercially available, for example under the trade name Neo Heliopan 303 from Symrise.

Octisalate is commercially available, for example under the trade name Neo Heliopan OS/BP from Symrise.

Bis-ethylhexyloxyphenol methoxyphenyl triazine (BEMT) is commercially available for example under the trade name Tinosorb® S from BASF.

The composition may contain additional UV absorbers or UV blockers as known in the art.

Examples of other UV absorbers include, but are not limited to: methoxycinnamate derivatives such as octyl methoxycinnamate and isoamyl methoxycinnamate; camphor derivatives such as 4-methyl benzylidene camphor, camphor benzalkonium methosulfate, and terephthalylidene dicamphor sulfonic acid; other salicylate derivatives; benzoic acid derivatives such as aminobenzoic acid and octyldimethyl para-amino benzoic acid; other β,β-diphenylacrylates; dioctyl butamido triazone; octyl triazone; menthyl anthranilate; triazone derivatives such as ethylhexyl triazone (Uvinul® T150); diethylhexyl butamido triazone (UVASorb® HEB); benzoate derivatives such as diethylamino hydroxybenzoyl hexyl benzoate (Uvinul® A Plus), benzotriazole derivatives such as drometrizole trisiloxane (Mexoryl® XL), methylene bis-benzotriazolyl tetramethylbutylphenol (Tinosorb® M); tris-biphenyl triazine; (2-{4-[2-(4-Diethylamino-2-hydroxy-benzoyl)-benzoyl]-piperazine-1-carbonyl}-phenyl)-(4-diethylamino-2-hydroxy-phenyl)-methanone; phenylbenzimidazole sulfonic acid (Eusolex® 232); merocyanine derivatives; bis(butylbenzoate) diaminotriazine aminopropylsiloxane; and bis-ethylhexyloxyphenol methoxyphenyl triazine encapsulated in a polymer matrix.

Similarly, the composition may optionally contain polymeric organic UV absorbers. As used herein, “polymeric organic UV absorber” means a polymeric compound comprising organic chromophores attached to a polymer chain. For instance, the polymer chain may be a polysiloxane having for example an average molecular weight of greater than 6000 Daltons. Examples of such polysiloxane UV absorbers include, without limitation, Parsol® SLX commercially available from DSM and polysilicone-15. These polysiloxanes absorb in the UVB (λₘₐₓ=312 nm) part of the spectrum and are typically combined with UVA filters to achieve broad-spectrum protection.

### Polymeric Emulsifier

The composition also contains a polymeric emulsifier selected from the group consisting of sodium acryloyldimethyltaurate/vinylpyrrolidine crosspolymer and acrylates/C10-30 alkyl acrylate crosspolymer. The composition may comprise a combination of such polymeric emulsifiers. The composition may contain about 0.01 to about 1.0 weight percent of polymeric emulsifier.

In one embodiment, the polymeric emulsifier is sodium acryloyldimethyltaurate/vinylpyrrolidine crosspolymer. Sodium acryloyldimethyltaurate/vinylpyrrolidine crosspolymer is commercially available for example as ARISTOFLEX® AVS from Clariant.

The composition may contain about 0.4 to about 0.6 weight percent of sodium acryloyldimethyltaurate/vinylpyrrolidine crosspolymer.

Alternatively, the polymeric emulsifier may be an acrylates/C10-30 alkyl acrylate crosspolymer. A commercially available acrylates/C10-30 alkyl acrylate crosspolymer is for example PEMULEN™ EZ-4U sold by Lubrizol Corporation.

The composition may contain about 0.05 to about 0.5 weight percent of an acrylate crosspolymer.

### Solid Absorbent

The composition also comprises a solid absorbent selected from the group consisting of silica and aluminum starch octenylsuccinate. The composition may comprise a combination of silica and aluminum starch octenylsuccinate.

The total weight of solid absorbent in the composition is about 2% to about 15%, preferably about 3.5% to about 12%, percent by weight based on the total weight of the composition.

The composition may comprise about 1.5% to about 10% weight percent silica. For compositions providing a mattifying effect or oil absorbing effect, about 4% to about 10% percent by weight silica may be used. For lightweight compositions, about 1.5% to about 2.5% percent by weight silica may be used.

The composition may comprise about 0.5% to about 7% percent by weight aluminum starch octenylsuccinate. For compositions providing a mattifying effect or oil absorbing effect, about 2% to about 7% percent by weight aluminum starch octenylsuccinate may be used. For lightweight compositions, about 0.5% to about 1% percent by weight aluminum starch octenylsuccinate may be used.

### Trilaureth-4 Phosphate

The composition further comprises trilaureth-4 phosphate. The composition may contain about 0.75% to about 3%, preferably about 2% to about 2.5%, weight percent of trilaureth-4 phosphate.

Trilaureth-4 phosphate is commercially available for example as HOSTAPHAT KL 340D from Clariant.

### Film Former

The composition may comprise one or more film formers. For example, the composition may comprise the film former tricontanyl PVP (tricontanyl povidone or 2-Pyrrolidinone, 1-ethenyl-, polymer with 1-triacontene).

The composition may comprise about 0.5 to about 4% by weight of triacontanyl PVP, preferably about 1 to about 3%, for example about 2%, by weight of triacontanyl PVP, based on the total weight of the composition.

Triacontanyl PVP is commercially available, for example from ISP Freetown Fine Chemicals Inc. under the trade name Antaron™ WP-660 polymer.

### Humectant

The composition may comprise a humectant. Examples of humectants include, without limitation to, glycerin, pentylene glycol, butylene glycol.

The composition may comprise glycerin. In particular, the composition may comprise about 1% to about 5% weight percent glycerin.

Compositions comprising at least about 3% weight percent of glycerin based on the total weight of the composition desirably provide hydrating but lightweight aesthetics.

For example, a preferred composition having a light weight, watery break may comprise of about 3.5 weight percent glycerin, about 1 weight percent aluminum starch octenylsuccinate, about 2.5 weight percent silica and about 2 weight percent trilaureth-4 phosphate. This composition may also additionally contain glyceryl stearate.

### SPF

The composition may provide a Sun Protection Factor (SPF) of at least about 30, preferably at least about 50, more preferably at least about 70. SPF may be measured in vivo or in vitro.

The SPF of the composition may be tested in vivo using the methodology set forth in the US FDA OTC monograph for sunscreens (U.S. Food and Drug Administration Over-the-Counter Monograph M020: Sunscreen Drug Products for Over-the-Counter Human Use (Posted Sep. 24, 2021); Final Administrative Order (OTC000006), effective upon enactment of the Coronavirus Aid, Relief, and Economic Security Act (CARES Act), Public Law 116-136, on Mar. 27, 2020) (“IN VIVO SPF TEST METHOD”).

In one embodiment, the composition has an SPF as measured by the IN VIVO SPF TEST METHOD of at least about 40. In another embodiment, the composition has an SPF as measured by the IN VIVO SPF TEST METHOD of at least about 50. In another embodiment, the composition has an SPF as measured by the IN VIVO SPF TEST METHOD of at least about 70.

Alternatively, the SPF of the composition may be tested using the following IN VITRO SPF TEST METHOD. The baseline transmission of a PMMA plate (substrate, available from Helioscience, Marseille, France) is measured for UV absorbance using calibrated Labsphere® UV-10005 UV transmission analyzer or a Labsphere® UV-2000S UV transmission analyzer (Labsphere, North Sutton, N.H., USA). A test sample is then applied to the PMMA plate using an application density of about 0.75 mg/cm² by rubbing into a uniform thin layer with the operator's finger. The sample is allowed to dry for 15 minutes and then measured for UV absorbance in the same way. The absorbance measures are used to calculate SPF as known in the art using the following equation:

$$SPF in vitro = \frac{{\int }_{λ = 290 n m}^{λ = 400 n m} E ( λ ) * I ( λ ) * d λ}{{\int }_{λ = 290 n m}^{λ = 400 n m} E ( λ ) * I ( λ ) * {10}^{{A}_{0} ( λ )} * d λ}$$

$$in which :$$

$$E ( λ ) = Erythema action spectrum ;$$

$$I ( λ ) = Spectral irradiance received from the UV source ;$$

$${A}_{0} ( λ ) = Mean monochromatic absorbance of the test product layer before UV exposure ; and$$

$$d ( λ ) = Wavelength step ( 1 nm ) .$$

In one embodiment, the composition has an SPF as measured by the IN VITRO SPF TEST METHOD of at least about 20, preferably at least about 30.

### Viscosity

Certain embodiments of the composition are advantageously lightweight and watery. Such compositions have a lower viscosity, for example a lower shear viscosity.

### Topical Composition

The sunscreen composition can be used by topically applying to a mammal, e.g., by the direct laying on, wiping or spreading of the composition on the skin, hair, or nails of a mammal, particularly a human.

The sunscreen composition is in the form of an oil-in-water emulsion containing a continuous water phase and a discontinuous oil phase dispersed within the continuous water phase. The composition of the invention is surprisingly and advantageously phase stable even though it may contain a relatively high level of solid UV filters or solid absorbents, for example about 2% percent by weight or greater based on the total weight of the composition.

The oil phase may be such that it is present in discrete droplets or units having an average diameter of about 0.5 micron to about 1000 microns, such as from about 1 micron to about 100 microns.

The percentage by weight of water phase included in the compositions may range from about 35 to about 70% by weight of the composition, such as from about 40% to about 65% by weight of the composition.

The sunscreen composition may be prepared using mixing and blending methodology well known in the sunscreen and cosmetic art. Typically, the sunscreen composition is produced in a one pot process.

The composition may be combined with a “cosmetically acceptable topical carrier,” i.e., a carrier for topical use that capable of containing the other ingredients dispersed or dissolved therein and possessing acceptable properties rendering it safe to use topically.

The composition may optionally comprise a wide variety of additional oil-soluble materials and/or water-soluble materials conventionally used in compositions for use on skin, at their art-established levels. For example, surfactants, pearlescent or opacifying agents, thickeners, emollients, conditioners, chelating agents, exfoliants, other oil absorbers, and additives that enhance the appearance, feel, or fragrance of the cleansing composition, such as colorants, fragrances, preservatives, pH adjusting agents, and the like, can be included, provided they do not significantly interfere with the stability or aesthetics of the composition.

Water soluble or water dispersible polymers may be added to the compositions. The water dispersible polymers are comprised of a water-insoluble polymer that is typically micronized and dispersed into a water carrier, possibly with the use of a surface active dispersing aid. The water dispersible polymers are capable of forming a film and improving water resistance of the compositions. Examples of water-soluble polymers include Polyaldo® 10-1-L (Polyglyceryl-10 Laurate), available from Lonza. Examples of water dispersible polymers include water dispersible polyurethanes, such as Baycusan® C1000 (Polyurethane-34), available from Bayer, Dow Corning® 2501 (Bis-PEG-18 Methyl Ether Dimethyl Silane), available from Dow Corning, Eastman AQTM 38S (Polyester-5), available from Eastman Chemical, and Intelimer® 8600 (C8-22 Alkyl Acrylates/Methacrylic Acid Crosspolymer) available from Air Products.

The sunscreen composition may also comprise additional film formers. Acceptable additional film formers include polyethylene, which is available from New Phase Technologies as Performalene® 400, a polyethylene having a molecular weight of 400. Another suitable film former is polyethylene 2000 (molecular weight of 2000), which is available from New Phase Technologies as Performalene®. Another suitable film former is synthetic wax, also available from New Phase Technologies as Performa® V-825. Other typical film-formers include VP/hexadecene copolymer, acrylates/acrylamide copolymer, acrylates copolymer, acrylates/C12-C22 alkylmethacrylate copolymer, polyethylene, waxes, VP/dimethiconylacrylate/polycarbamylpolyglycol ester, butylated PVP, PVP/hexadecene copolymer, octadecene/MA copolymer, PVP/eicosene copolymer, tricontanyl PVP, Brassica Campestris/Aleuritis Fordi Oil copolymer, decamethyl cyclopentasiloxane (and) trimethylsiloxysilicate, Dimethicone; Acrylates/Dimethicone Copolymer and mixtures thereof. In some cases, the film former is acrylates/C12-C22 alkylmethacrylate copolymer sold under the tradename Allianz™ OPT by Ashland.

Suitable emollients for use in the composition include mineral oils, petrolatum, vegetable oils (e.g. triglycerides such as caprylic/capric triglyceride, Helianthus Annuus (Sunflower) Seed Oil), waxes and other mixtures of fatty esters, including but not limited to esters of glycerol (e.g, isopropyl palmitate, isopropyl myristate, diisopropyl adipate, dicaprylyl carbonate, c12-15 alkyl benzoate, isononyl isononanoate, neopentyl glycol diheptonoate, neopentyl glycol dibenzoate, butyloctyl salicylate, ethylhexylmethoxycrylene), hydrocarbons (e.g, isododecane, isohexadecane, squalane), ethers (e.g, PPG-15 stearyl ether, dicaprylyl ether), silicone oils such as dimethicone, and jojoba esters. In certain embodiments, mixtures of triglycerides (e.g. caprylic/capric triclycerides) and esters of glycols (e.g. isopropyl myristate).

The sunscreen composition may comprise one or more additional emulsifiers, such as PEG-100 stearate, glyceryl stearate, or potassium cetyl phosphate, and fatty alcohols, such as cetyl alcohol.

Examples of suitable solvents include propylene glycol, polyethylene glycol, polypropylene glycol, glycerol, 1,2,4-butanetriol, sorbitol esters, 1,2,6-hexanetriol, ethanol, and mixtures thereof.

In certain embodiments, the composition includes a pigment suitable for providing color or hiding power. The pigment may be one suitable for use in a color cosmetic product, including compositions for application to the hair, nails and/or skin, especially the face. Color cosmetic compositions include, but are not limited to, foundations, concealers, primers, blush, mascara, eyeshadow, eyeliner, lipstick, nail polish and tinted moisturizers. The pigment suitable for providing color or hiding power may be composed of iron oxides, including red and yellow iron oxides, titanium dioxide, ultramarine and chromium or chromium hydroxide colors, and mixtures thereof. The pigment may be a lake pigment, e.g. an organic dye such as azo, indigoid, triphenylmethane, anthraquinone, and xanthine dyes that are designated as D&C and FD&C blues, browns, greens, oranges, reds, yellows, etc., precipitated onto inert binders such as insoluble salts. Examples of lake pigments include Red #6, Red #7, Yellow #5, Violet #2 and Blue #1. The pigment may be an interference pigment. Examples of interference pigments include those containing mica substrates, bismuth oxycloride substrates, and silica substrates, for instance mica/bismuth oxychloride/iron oxide pigments commercially available as CHROMALITE pigments (BASF), titanium dioxide and/or iron oxides coated onto mica such as commercially available FLAMENCO pigments (BASF), mica/titanium dioxide/iron oxide pigments including commercially available KTZ pigments (Kobo products), CELLINI pearl pigments (BASF), and borosilicate-containing pigments such as REFLECKS pigments (BASF).

The composition may comprise one or more SPF boosters, for example styrene/acrylates copolymer, butyloctyl salicylate, or ethylhexyl methoxycrylene. The composition may comprise both styrene/acrylates copolymer and ethylhexyl methoxycrylene.

The composition may comprise one or more preservatives, for example chlorphenesin, phenoxyethanol, or hydroxyacetophenone. In a preferred embodiment, the composition contains a combination of all three. For example, the composition may comprise about 0.27% by weight chlorphenesin, about 0.6% by weight phenoxyethanol, and about 0.5% by weight hydroxyacetophenone, which has been found to provide a particularly efficacious preservative package for the compositions of the invention.

In another embodiment, the sunscreen composition has a pH of about 5.0 to about 7.0, for example about 6.0.

The composition may further comprise one or more other cosmetically acceptable active agents include for example anti-acne agents, shine control agents, anti-microbial agents, anti-inflammatory agents, anti-mycotic agents, anti-parasite agents, external analgesics, antioxidants, keratolytic agents, moisturizers, nutrients, vitamins, energy enhancers, anti-perspiration agents, astringents, deodorants, firming agents, anti-callous agents, and agents for skin conditioning.

The amount of other cosmetically active agents may range from about 0.001% to about 20% by weight of the composition, e.g., about 0.005% to about 10% by weight of the composition, such as about 0.01% to about 5% by weight of the composition.

The cosmetically acceptable active agent may be selected for instance from D-panthenol carotenoids, ceramides, polyunsaturated fatty acids, essential fatty acids, enzymes such as laccase, enzyme inhibitors, minerals, steroids such as hydrocortisone, 2-dimethylaminoethanol, copper salts such as copper chloride, peptides like argireline, syn-ake, acetyl dipeptide-31 amide and those containing copper, coenzyme Q10, amino acids such as proline, vitamins, lactobionic acid, acetyl-coenzyme A, niacin, riboflavin, thiamin, ribose, electron transporters such as NADH and FADH2, natural extracts such as from aloe vera, feverfew (Chrysanthemum Parthenium (Feverfew) Flower/Leaf/Steam Juice), oatmeal, dill, blackberry, princess tree, Picia anomala, and chicory, resorcinols such as 4-hexyl resorcinol, curcuminoids, sugar amines such as N-acetyl glucosamines, bisabolol, and derivatives and mixtures thereof.

Examples of vitamins and their derivatives include, but are not limited to, vitamin A, vitamin B's such as vitamin B3, niacinamide, vitamin B5, and vitamin B12, vitamin C, vitamin K, and different forms of vitamin E such as alpha, beta, gamma or delta tocopherols or their mixtures, and derivatives thereof.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, propolis and Zingiber Officinale (Ginger) Root Extract.

In one embodiment, the composition comprises a cosmetically acceptable active agent selected from the group consisting of hyaluronic acid, ascorbic acid, tocopheryl acetate, niacinamide, green tea extract, bisabolol and combinations thereof.

The following examples further illustrate the invention.

### Example 1

The compositions set forth in Tables 1-4 were made according to the invention.

TABLE 1
Compositions 1 and 2

| | Composition 1 | Composition 2 | |
|---|---|---|---|
| US INCI Name | Percentage (% w/w) | Percentage (% w/w) | Function |
| Water | 49.427 | 53.027 | Vehicle |
| Disodium EDTA | 0.20 | 0.20 | Chelating Agent |
| Glycerin | 3.5 | 0 | Humectant |
| Sodium Acryloyldimethyltaurate/ | 0.5 | 0.5 | Viscosity Increasing |
| VP Crosspolymer | | | Agent |
| Avobenzone | 3 | 3 | Sunscreen Agent |
| Homosalate | 13.5 | 15 | Sunscreen Agent |
| Octisalate | 5 | 5 | Sunscreen Agent |
| Octocrylene | 10 | 0 | Sunscreen Agent |
| Bis-ethylhexyloxyphenol | 0 | 6 | Sunscreen Agent |
| methoxyphenyl triazine | | | |
| Glyceryl Stearate | 0.3 | 0.3 | Emulsifier |
| Phenoxyethanol | 0.6 | 0.6 | Preservative |
| Chlorphenesin | 0.27 | 0.27 | Preservative |
| Aluminum Starch Octenylsuccinate | 1 | 1 | Absorbent |
| Silica | 2.5 | 2.5 | Absorbent |
| Butyloctyl Salicylate | 5 | 5 | Emollient |
| C12-15 Alkyl Benzoate | 0 | 3 | Emollient |
| Dimethicone | 0.6 | 0 | Emollient |
| Trilaureth-4 Phosphate | 2 | 2 | Emulsifier |
| Triacontanyl PVP | 2 | 2 | Film Former |
| Hydroxyacetophenone | 0.5 | 0.5 | Skin Conditioner |
| Tocopheryl Acetate | 0.1 | 0.1 | Antioxidant |
| Sodium Hydroxide | 0.003 | 0.003 | pH Adjuster |
| Total | 100.00 | 100.00 | |

TABLE 2
Compositions 3 and 4

| | Composition 3 | Composition 4 |
|---|---|---|
| | Percentage | Percentage |
| US INCI Name | (% w/w) | (% w/w) |
| Water | 49.82 | 48.62 |
| Disodium EDTA | 0.10 | 0.10 |
| Glycerin | 3.50 | 3.50 |
| Hydrolyzed Hyaluronic Acid | 0.05 | 0.05 |
| Hydroxyacetophenone | 0.5 | 0.50 |
| Chlorphenesin | 0.27 | 0.27 |
| Sodium Acryloyldimethyltaurate/ | 0.50 | 0.50 |
| VP Crosspolymer | | |
| Avobenzone | 3.00 | 3.00 |
| Homosalate | 13.50 | 13.50 |
| Octisalate | 5.00 | 5.00 |
| Octocrylene | 10.00 | 10.00 |
| Butyloctyl Salicylate | 5.00 | 5.00 |
| Glyceryl Stearate | 0.30 | 0.30 |
| Phenoxyethanol | 0.60 | 0.60 |
| Aluminum Starch Octenylsuccinate | 1.00 | 1.00 |
| Silica | 2.50 | 2.50 |
| Caprylic/Capric Triglyceride; | 1 | 1.00 |
| Hydroxymethoxyphenyl Decanone | | |
| Ascorbic Acid | 0.05 | 0.05 |
| Tocopheryl Acetate | 0.1 | 0.10 |
| Dimethicone | 0.4 | 0.60 |
| Trilaureth-4 Phosphate | 2.00 | 2.00 |
| Triacontanyl PVP | 1.80 | 1.80 |
| Sodium Hydroxide | 0.008 | 0.010 |
| Total: | 100.00 | 100.00 |

TABLE 3
Composition 5

| US INCI Name | Percentage (% w/w) | Function |
|---|---|---|
| Water | 42.61 | Solvent |
| Disodium EDTA | 0.10 | Chelating Agent |
| Chlorphenesin | 0.27 | Preservative |
| Niacinamide | 0.10 | Skin Conditioner |
| Acrylates/C10-30 Alkyl | .2 | Emulsion Stabilizer |
| Acrylate Crosspolymer | | |
| Avobenzone | 3.00 | Sunscreen Agent |
| Octisalate | 5.00 | Sunscreen Agent |
| Octocrylene | 10.00 | Sunscreen Agent |
| Homosalate | 15.00 | Sunscreen Agent |
| Cetyl Alcohol | 1.00 | Emulsifier |
| Trilaureth-4 Phosphate | 2.50 | Emulsifier |
| Triacontanyl PVP | 1.80 | Film Former |
| Butyloctyl Salicylate | 5.00 | Skin Conditioner |
| Tocopheryl Acetate | 0.10 | Antioxidant |
| Aluminum Starch | 5.00 | Absorbent |
| Octenylsuccinate | | |
| Silica | 4.00 | Absorbent |
| Silica | 3.00 | Absorbent |
| Camellia Sinensis Leaf | 0.2 | Skin Conditioner |
| Extract; Butylene Glycol; | | |
| Water | | |
| Hydroxyacetophenone | 0.50 | Skin Conditioner |
| Phenoxyethanol | 0.6 | Preservative |
| Citric Acid | 0.01 | pH Adjuster |
| Sodium Hydroxide | 0.01 | pH Adjuster |
| Total | 100.00 | |

TABLE 4
Compositions 6, 7, 8, 9

| | | Percentage | Percentage | Percentage | Percentage |
|---|---|---|---|---|---|
| | | (% w/w) | (% w/w) | (% w/w) | (% w/w) |
| US INCI name | Function | Composition 6 | Composition 7 | Composition 8 | Composition 9 |
| Water | SOLVENT | 49.43 | 48.93 | 49.02 | 48.32 |
| Butyloctyl Salicylate | SKIN CONDITIONER | 5.00 | 5.00 | 5.00 | 5.00 |
| Aluminum Starch | ABSORBENT | 1.00 | 1.00 | 1.00 | 1.00 |
| Octenylsuccinate | | | | | |
| Avobenzone | SUNSCREEN AGENT | 3.00 | 3.00 | 3.00 | 3.00 |
| Disodium EDTA | CHELATING AGENT | 0.200 | 0.200 | 0.20 | 0.20 |
| Octisalate | SUNSCREEN AGENT | 5.00 | 5.00 | 5.00 | 5.00 |
| Glyceryl Stearate | EMULSIFIER | 0.30 | 0.30 | 0.30 | 0.30 |
| Homosalate | SUNSCREEN AGENT | 13.50 | 13.50 | 13.50 | 13.50 |
| Octocrylene | SUNSCREEN AGENT | 10.00 | 10.00 | 10.00 | 10.00 |
| Tocopheryl Acetate | ANTIOXIDANT | 0.10 | 0.10 | 0.20 | 0.10 |
| Sodium Hydroxide | PH ADJUSTER | 0.003 | 0.003 | 0.003 | 0.003 |
| Trilaureth-4 Phosphate | EMULSIFIER | 2.00 | 2.00 | 2.00 | 2.00 |
| Triacontanyl PVP | FILM FORMING AGENT | 2.00 | 2.00 | 2.00 | 2.00 |
| Dimethicone | EMOLLIENT | 0.60 | 0.60 | 0.60 | 0.60 |
| Chlorphenesin | PRESERVATIVE | 0.27 | 0.27 | 0.27 | 0.27 |
| Silica | BULKING AGENT | 2.50 | 2.50 | 2.50 | 2.50 |
| Glycerin | HUMECTANT | 3.50 | 3.50 | 3.50 | 3.50 |
| Phenoxyethanol | PRESERVATIVE | 0.60 | 0.60 | 0.60 | 0.60 |
| Hydroxyacetophenone | SKIN CONDITIONER | 0.50 | 0.50 | 0.50 | 0.50 |
| Sodium | VISCOSITY | 0.50 | 0.50 | 0.50 | 0.50 |
| Acryloyldimethyltaurate/ | INCREASING | | | | |
| VP Crosspolymer | | | | | |
| Ascorbyl Palmitate | ANTIOXIDANT | | | | 0.10 |
| Avena Sativa (Oat) | Soothing agent | | | | 1.00 |
| Kernel Extract; Water; | | | | | |
| Glycerin | | | | | |
| Zingiber Officinale | SKIN CONDITIONER | | | 0.20 | |
| (Ginger) Root Extract | | | | | |
| Yellow 5 | COLORANT | | | 0.003 | |
| Red 40 | COLORANT | | | 0.001110 | |
| Camellia Sinensis | SKIN CONDITIONER | | 0.10 | | |
| Leaf Extract; Butylene | | | | | |
| Glycol; Water | | | | | |
| Bisabolol | SKIN CONDITIONER | | 0.40 | | |
| Chrysanthemum Parthenium | SKIN CONDITIONER | | | 0.10 | |
| (Feverfew) Extract | | | | | |
| SPF (in-vitro) | — | 34.3731 | 35.4458 | 34.2318 | 35.6994 |

### Example 2

The following compositions were made and tested for in vitro SPF using the IN VITRO SPF TEST METHOD. Compositions 10, 11 and 14 were made according to the invention, and Compositions 12 and 13 were comparative.

| | | Percentage (% w/w) | | | | |
|---|---|---|---|---|---|---|
| | | Composition | Composition | Composition | Composition | Composition |
| INCI Name | Function | 10 | 11 | 12 | 13 | 14 |
| Octisalate | Sun Filter | 5.00 | 5.00 | 5.00 | 5.00 | 5.00 |
| Avobenzone | Sun Filter | 3.00 | 3.00 | 3.00 | 3.00 | 3.00 |
| Homosalate | Sun Filter | 13.50 | — | — | — | — |
| Octocrylene | Sun Filter | 10.00 | | | | |
| BEMT | Sun Filter | — | 6.00 | 6.00 | 6.00 | 6.00 |
| Butyloctyl Salicylate | Emollient | 5.00 | — | — | — | — |
| Dimethicone | Emollient | 0.60 | — | — | — | — |
| C12-15 Alkyl | Emollient | — | 12.00 | 10.00 | 10.00 | 10.00 |
| Benzoate | | | | | | |
| Cetyl Dimethicone | Slip Modifier | — | 0.75 | 0.75 | 0.50 | 0.75 |
| Caprylyl Methicone | Slip Modifier | — | — | 1.00 | 1.00 | — |
| Trilaureth 4- | Emulsifier | 2.00 | 2.00 | — | — | 2.00 |
| Phosphate | | | | | | |
| Glycerl Stearate | Emulsifier | 0.30 | 0.30 | — | — | 0.30 |
| PEG-100 Stearate; | Emulsifier | — | — | 1.70 | 1.70 | — |
| Glyceryl Stearate | | | | | | |
| Potassium Cetyl | Emulsifier | — | — | 1.40 | 1.20 | — |
| Phosphate | | | | | | |
| Sodium | Thickening | 0.50 | 0.45 | — | — | 0.45 |
| Acryloyldimethyl- | Agent | | | | | |
| taurate/ VP | | | | | | |
| Crosspolymer | | | | | | |
| Xanthan Gum | Thickening | — | — | 0.25 | 0.30 | — |
| | Agent | | | | | |
| Sodium | Thickening | — | — | 0.33 | 0.45 | — |
| Polyacrylate; | Agent | | | | | |
| Ethylhexyl Stearate; | | | | | | |
| Trideceth-6 | | | | | | |
| Disodium EDTA | Chelating | 0.20 | 0.20 | 0.20 | 0.20 | 0.20 |
| | Agent | | | | | |
| Chlorphenesin | Preservative | 0.27 | 0.27 | 0.25 | 0.25 | 0.27 |
| Hydroxyaceto- | Skin | 0.50 | 0.50 | — | — | 0.50 |
| phenone | Conditioner | | | | | |
| Phenoxyethanol | Preservative | 0.60 | 0.60 | 0.70 | 0.70 | 0.60 |
| Ethylhexylglycerin | Skin | — | — | 0.50 | 0.50 | — |
| | Conditioner | | | | | |
| Caprylyl Glycol | Skin | — | — | 0.60 | 0.60 | — |
| | Conditioner | | | | | |
| Water | Solvent | 48.32 | 60.32 | 59.97 | 62.00 | 62.33 |
| Glycerin | Humectant | 3.50 | 3.50 | 3.50 | 3.50 | 3.50 |
| Tocopheryl Acetate | Antioxidant | 0.10 | 0.10 | 0.10 | — | 0.10 |
| Ascorbyl Palmitate | Antioxidant | 0.10 | — | — | — | — |
| Avena Sative (Oat) | Soothing | 1.00 | — | — | — | — |
| Kernel Extract; | Agent | | | | | |
| Water; Glycerin | | | | | | |
| Triacontanyl PVP | Film Former | 2.00 | 2.00 | — | — | 2.00 |
| Dimethicone; | Film Former | — | — | 1.00 | — | — |
| Acrylates/Dime- | | | | | | |
| thicone Copolymer | | | | | | |
| Acrylates/C12-22 | Film Former | — | — | — | 0.50 | — |
| Alkyl Methacrylate | | | | | | |
| Copolymer | | | | | | |
| Hydrolyzed Jojoba | Emollient | — | — | 0.25 | 0.50 | — |
| Esters; Jojoba | | | | | | |
| Esters; Water | | | | | | |
| Hydrogenated | Oil | — | — | 0.75 | — | — |
| Castor Oil | Thickener | | | | | |
| Copernicia Cerifera | Oil | — | — | 0.75 | — | — |
| (Carnauba) Wax | Thickener | | | | | |
| Synthetic Beeswax | Oil | — | — | — | 0.50 | — |
| | Thickener | | | | | |
| Behenyl Alcohol | Oil | — | — | — | 0.40 | — |
| | Thickener | | | | | |
| Silica | Absorbent | 2.50 | 2.00 | — | — | 2.00 |
| Silica | Absorbent | — | — | 1.00 | 1.20 | — |
| Aluminum Starch | Absorbent | 1.00 | 1.00 | 1.00 | — | 1.00 |
| Octenylsuccinate | | | | | | |
| Sodium Hydroxide | pH Adjustor | 0.01 | 0.008 | — | — | — |
| In-Vitro SPF | — | 36.2 | 19.9 | 12.5 | 6.0 | 24.9 |

1. A sunscreen composition comprising:
(i) an organic UV filter selected from the group consisting of avobenzone, homosalate, octocrylene, octisalate, bis-ethylhexyloxyphenol methoxyphenyl triazine, and combinations thereof;
(ii) a polymeric emulsifier selected from the group consisting of sodium acryloyldimethyltaurate/VP crosspolymer, acrylates/C10-30 alkyl acrylate crosspolymer, and combinations thereof;
(iii) a solid absorbent selected from the group consisting of silica, aluminum starch octenylsuccinate, and combinations thereof; and
(iv) trilaureth-4 phosphate;
wherein the composition is an oil-in-water emulsion.

2. The composition of claim 1, comprising:
(i) up to 5 weight % of avobenzone based on the total weight of the composition;
(ii) up to 15 weight % of homosalate based on the total weight of the composition;
(iii) up to 10 weight % of octocrylene based on the total weight of the composition;
(iv) up to 5 weight % of octisalate based on the total weight of the composition;
(v) about 0.01 to about 1.0 weight % of polymeric emulsifier based on the total weight of the composition;
(vi) about 2 to about 15 weight % of solid absorbent based on the total weight of the composition; and
(vii) about 0.75 to about 3 weight % of trilaureth-4 phosphate based on the total weight of the composition.

3. The composition of claim 1, further comprising a humectant selected from the group consisting of glycerin, pentylene glycol, butylene glycol and combinations thereof.

4. The composition of claim 1, further comprising triacontanyl PVP.

5. The composition of claim 1, further comprising a cosmetically acceptable active agent selected from the group consisting of hyaluronic acid, ascorbic acid, tocopheryl acetate, niacinamide, green tea extract, and combinations thereof.

6. A sunscreen composition comprising:
(i) about 3 weight % of avobenzone based on the total weight of the composition;
(ii) about 15 weight % of homosalate based on the total weight of the composition;
(iii) about 10 weight % of octocrylene based on the total weight of the composition;
(iv) about 5 weight % of octisalate based on the total weight of the composition;
(v) about 0.2 weight % of acrylates/C10-30 alkyl acrylate crosspolymer based on the total weight of the composition;
(vi) about 7 weight % of silica based on the total weight of the composition;
(vii) about 5 weight % of aluminum starch octenylsuccinate based on the total weight of the composition; and
(viii) about 2.5 weight % of trilaureth-4 phosphate based on the total weight of the composition;
wherein the composition is an oil-in-water emulsion.

7. A sunscreen composition comprising:
(i) about 3 weight % of avobenzone based on the total weight of the composition;
(ii) about 13.5 weight % of homosalate based on the total weight of the composition;
(iii) about 10 weight % of octocrylene based on the total weight of the composition;
(iv) about 5 weight % of octisalate based on the total weight of the composition;
(v) about 0.5 weight % of sodium acryloyldimethyltaurate/vinylpyrrolidine crosspolymer based on the total weight of the composition;
(vi) about 2.5 weight % of silica based on the total weight of the composition;
(vii) about 1 weight % of aluminum starch octenylsuccinate based on the total weight of the composition;
(viii) about 2 weight % of trilaureth-4 phosphate based on the total weight of the composition; and
(ix) about 3.5 weight % of glycerin based on the total weight of the composition;
wherein the composition is an oil-in-water emulsion.

8. The sunscreen composition of claim 7 further comprising a cosmetically acceptable active agent selected from the group consisting of hyaluronic acid, ascorbic acid, tocopheryl acetate, niacinamide, green tea extract, and combinations thereof.

9. A sunscreen composition comprising:
(i) about 3 weight % of avobenzone based on the total weight of the composition;
(ii) about 5 weight % of octisalate based on the total weight of the composition;
(iii) about 6 weight % of bis-ethylhexyloxyphenol methoxyphenyl triazine based on the total weight of the composition,
(iv) about 0.45 weight % of sodium acryloyldimethyltaurate/vinylpyrrolidine crosspolymer based on the total weight of the composition;
(v) about 2 weight % of silica based on the total weight of the composition;
(vi) about 1 weight % of aluminum starch octenylsuccinate based on the total weight of the composition; and
(vii) about 2 weight % of trilaureth-4 phosphate based on the total weight of the composition;
wherein the composition is an oil-in-water emulsion.

10. The sunscreen composition of claim 9 further comprising about 2 weight % triacontanyl PVP based on the total weight of the composition.

11. A sunscreen composition comprising:
(i) about 3 weight % of avobenzone based on the total weight of the composition;
(ii) about 5 weight % of octisalate based on the total weight of the composition;
(iii) about 15 weight % of homosalate based on the total weight of the composition;
(iv) about 6 weight % of bis-ethylhexyloxyphenol methoxyphenyl triazine based on the total weight of the composition,
(v) about 0.5 weight % of sodium acryloyldimethyltaurate/vinylpyrrolidine crosspolymer based on the total weight of the composition;
(vi) about 2 weight % of silica based on the total weight of the composition;
(vii) about 1 weight % of aluminum starch octenylsuccinate based on the total weight of the composition; and
(viii) about 2 weight % of trilaureth-4 phosphate based on the total weight of the composition;
wherein the composition is an oil-in-water emulsion.

12. The sunscreen composition of claim 11 further comprising about 2 weight % triacontanyl PVP based on the total weight of the composition.
